(19) United States
(12) Patent Application Publication
(10) Pub. No.: US 2026/0022323 A1
(43) Pub. Date: Jan. 22, 2026

(54) INFORMATION PROCESSING DEVICE AND INFORMATION PROCESSING METHOD

(71) Applicant: CANON KABUSHIKI KAISHA, Tokyo (JP)

(72) Inventor: YASUHIRO HORIGUCHI, Kanagawa (JP)

(21) Appl. No.: 19/270,147

(22) Filed: Jul. 15, 2025

(30) Foreign Application Priority Data
Jul. 19, 2024 (JP) 2024-115612

(51) Int. Cl. C12M 1/36; C12M 1/12; C12M 1/26; C12M 1/34

(52) U.S. Cl. CPC C12M 41/48; C12M 25/00; C12M 33/08; C12M 41/36

(57) ABSTRACT

An information processing device includes a cell information acquisition unit configured to acquire information about a cells, a detachment condition acquisition unit configured to acquire information about a detachment condition for detaching the cell adhering to a culture substrate from the culture substrate by an external force being applied to the culture substrate, and a storage unit configured to store the information about the cell and the information about the detachment condition in association with each other.

### BACKGROUND

### Field of the Technology

The present disclosure relates to an information processing device and an information processing method for detaching cells from a culture substrate.

### Description of the Related Art

With the advancement of regenerative medicine, cell culture techniques using culture plates, petri dishes, or the like have been developed for medical treatments and research and development in the field of medicine. After cultivation, the cells adhere to the bottom surface of the vessel. Thus, it is necessary to detach the cells from the culture vessel to retrieve them. Methods for detaching such cells include the use of detachment enzymes or chemicals that act on the cell membrane, temperature-responsive polymers, and the application of ultrasonic waves to impart vibrational energy to the cells, thereby detaching them from the vessel. It is also common to combine and use a plurality of detachment methods. International Patent Publication No. WO 2019/111310 discusses a technique for detaching cells by applying ultrasonic waves to the cells within a petri dish from the outside. Japanese Patent Application Laid-Open No. 2014-113133 discusses a cell detachment device that causes a culture vessel held on a holder to reciprocate and collide with a collided member together with the holder, and the collision impact and vibration allow sample cells to be detached.

With the increasing number and variety of cells to be subjected to detachment, the associated detachment conditions also become more numerous. Consequently, it may become challenging to accurately track the specific detachment conditions applied to the detached cells.

### SUMMARY

According to an aspect of the present disclosure, an information processing device includes a cell information acquisition unit configured to acquire information about a cells, a detachment condition acquisition unit configured to acquire information about a detachment condition for detaching the cell adhering to a culture substrate from the culture substrate by an external force being applied to the culture substrate, and a storage unit configured to store the information about the cell and the information about the detachment condition in association with each other.

According to another aspect of the present disclosure, an information processing method includes acquiring information about a cell, acquiring information about a detachment condition for detaching the cell adhering to a culture substrate from the culture substrate by an external force to be applied to the culture substrate, and storing the information about the cell and the information about the detachment condition in association with each other.

Features of the present disclosure will become apparent from the following description of embodiments with reference to the attached drawings. The following description of embodiments is described by way of example.

### BRIEF DESCRIPTION OF THE DRAWINGS

FIG. 1 is a block diagram illustrating an information processing device according to an exemplary embodiment of the present disclosure.

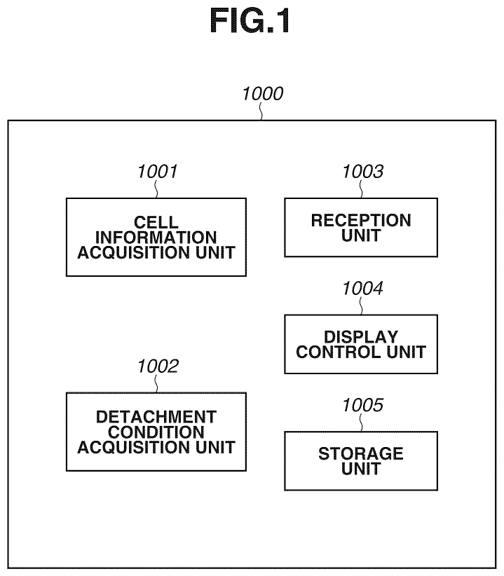

FIG. 2 is a block diagram illustrating a cell detachment system according to an exemplary embodiment of the present disclosure.

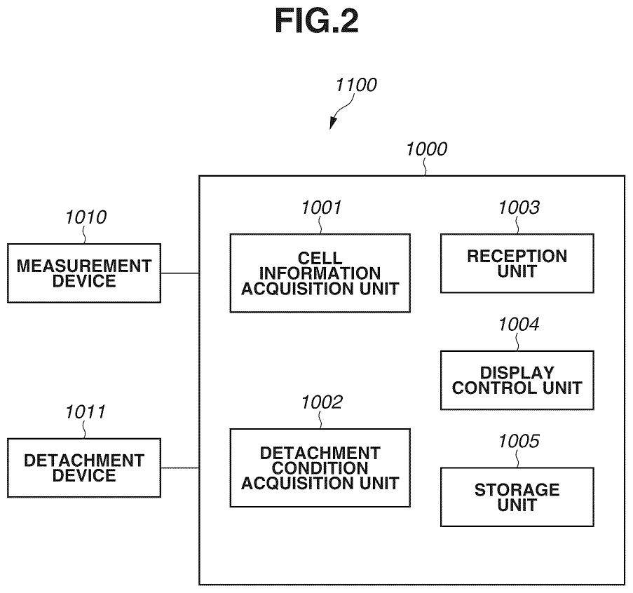

FIG. 3 is a block diagram illustrating a hardware configuration of the information processing device according to an exemplary embodiment of the present disclosure.

FIG. 4 is a flowchart illustrating an information processing method according to an exemplary embodiment of the present disclosure.

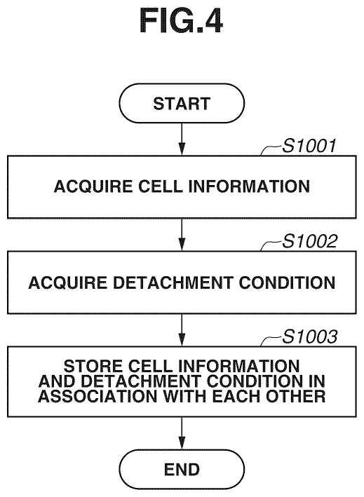

FIG. 5 is an overview diagram of a cell detachment system according to an exemplary embodiment of the present disclosure.

FIG. 6 illustrates an example of an operation screen of a graphical user interface (GUI) according to an exemplary embodiment of the present disclosure.

FIG. 7 illustrates an example of a GUI operation screen to be displayed after each state duration is confirmed according to an exemplary embodiment of the present disclosure.

FIG. 8 illustrates temporal changes in an indicator position on each state display bar according to an exemplary embodiment of the present disclosure.

FIG. 9 illustrates temporal changes in an indicator position on each state display bar according to an exemplary embodiment of the present disclosure.

FIG. 10 illustrates temporal changes in an indicator position on each state display bar according to an exemplary embodiment of the present disclosure.

FIG. 11 illustrates temporal changes in an indicator position on each state display bar according to an exemplary embodiment of the present disclosure.

FIG. 12 illustrates temporal changes in an indicator position on each state display bar according to an exemplary embodiment of the present disclosure.

FIG. 13 is a schematic diagram illustrating a GUI operation screen according to an exemplary embodiment of the present disclosure.

FIG. 14 is a schematic diagram illustrating a GUI operation screen according to an exemplary embodiment of the present disclosure.

FIG. 15 is a flowchart illustrating a detachment operation and a detachment condition storage operation according to an exemplary embodiment of the present disclosure.

FIG. 16 is a schematic diagram illustrating a vibration condition setting screen according an exemplary embodiment of the present disclosure.

FIG. 17 is a schematic diagram illustrating a shaking condition setting screen according to an exemplary embodiment of the present disclosure.

FIG. 18 is a schematic diagram illustrating a tapping condition setting screen according to an exemplary embodiment of the present disclosure.

FIG. 19 is a schematic diagram illustrating a content of a detachment condition stored file according to an exemplary embodiment of the present disclosure.

FIG. 20 is a schematic diagram illustrating a content of a detachment condition stored file according to an exemplary embodiment of the present disclosure.

FIG. 21 is a schematic diagram illustrating a detachment condition setting screen according to an exemplary embodiment of the present disclosure.

FIG. 22 is a schematic diagram illustrating a detachment condition setting screen according to an exemplary embodiment of the present disclosure.

FIG. 23 is a schematic diagram illustrating a detachment condition setting screen according to an exemplary embodiment of the present disclosure.

FIG. 24 is a schematic diagram illustrating a detachment condition setting screen according to an exemplary embodiment of the present disclosure.

FIG. 25 is a schematic diagram illustrating a detachment condition setting screen according to an exemplary embodiment of the present disclosure.

FIG. 26 is a schematic diagram illustrating a wizard screen to be displayed upon selection of a detachment condition file according to an exemplary embodiment of the present disclosure.

FIG. 27 is a schematic diagram illustrating a wizard screen to be displayed upon selection of a detachment condition file according to an exemplary embodiment of the present disclosure.

### DESCRIPTION OF THE EMBODIMENTS

An information processing device and a cell detachment system according to exemplary embodiments of the present disclosure will be described in detail below with reference to the accompanying drawings. However, the configurations described in the following exemplary embodiments are merely examples, and the scope of the present disclosure is not limited by the configurations described in the exemplary embodiments.

### First Exemplary Embodiment

### Information Processing Device

An information processing device 1000 according to a first exemplary embodiment of the present disclosure includes a cell information acquisition unit 1001 that acquires information about cells, and a detachment condition acquisition unit 1002 that acquires information about detachment conditions for detaching the cells adhering to a culture substrate from the culture substrate. The information about the detachment conditions refers to the conditions for detaching the cell from the culture substrate through application of an external force to the culture substrate. The information processing device 1000 according to the first exemplary embodiment includes a storage unit 1005 that stores the information about the cell and the information about the detachment conditions in association with each other. Storing the information about the cell and the information about the detachment conditions in association with each other as described above makes it possible to easily recognize the detachment conditions under which the cell has been detached.

The information processing device 1000 according to the present exemplary embodiment may include a reception unit 1003 that receives the input of detachment conditions, and a display control unit 1004 that controls display of detachment conditions.

### (Application of External Force)

Application of an external force according to the present exemplary embodiment may be performed by any of, for example, tapping on the culture substrate, shaking the culture substrate, or application of vibration to the culture substrate. The application of vibrations to the culture substrate may involve generating ultrasonic waves to apply vibrations in the ultrasonic frequency range to the culture substrate.

(Information about Culture Conditions)

In the present exemplary embodiment, the information about the detachment conditions includes at least one piece of information selected from the group consisting of information about an intensity of vibration to be applied to the culture substrate, information about a frequency of vibration to be applied to the culture substrate, and information about a duration of vibration to be applied to the culture substrate. Here, the information about the intensity of the vibration to be applied to the culture substrate (culture vessel) includes information about a voltage to be applied to an ultrasonic transducer that generates ultrasonic waves.

The information about the detachment conditions according to the present exemplary embodiment may be obtained based on information about a detachment result of cell detachment from the culture substrate. The information about the detachment result includes at least one piece of information selected from the group consisting of information about a detachment rate of cells, information about a survival rate of detached cells, information about a growth rate of detached cells, and information about functionality of detached cells.

The information about the detachment conditions according to the present exemplary embodiment may include at least one of the following: strength, frequency, and duration of tapping to be applied to the culture substrate; and amplitude, frequency, and duration of shaking to be applied to the culture substrate.

In a case where a detachment solution is used to detach cells from a culture substrate, information related to the detachment solution may be included in the detachment condition information. In a case where an acoustic matching material is provided between the culture substrate and the ultrasonic transducer for generating ultrasonic waves, information about the acoustic matching material may be included in the information about the detachment conditions.

(Information about Cells)

The information related to cells according to the present exemplary embodiment includes information about cell type.

(Information about Culture Conditions)

The storage unit 1005 as described above may store information about the cells, the information about the detachment conditions, and information about culture conditions for the cells in association with each other. Here, the information about culture conditions includes at least one piece of information selected from the group consisting of: information about cell seeding density, information about the culture medium, information about the culture duration, and information about the culture environment.

### (Display Control Unit)

According to the present exemplary embodiment, a display control unit that controls a display unit to display a driving state of a detachment device may be included. The driving state may include information about temporal changes in the driving state of the detachment device. The display control unit according to the present exemplary embodiment may control the display unit to display driving conditions for the detachment device, and may control the display unit to display the driving state and the driving conditions in association with each other.

### (Cell Detachment System)

A cell detachment system 1100 according to the present exemplary embodiment includes the information processing device 1000 according to the present exemplary embodiment described above, and a detachment device 1011 that detaches the cells adhering to the culture substrate from the culture substrate by applying an external force to the culture substrate. The detachment device 1011 includes a tapping unit for tapping the culture substrate, a shaking unit for shaking the culture substrate, and a vibration generation unit for applying vibrations to the culture substrate. In a case where the vibration generation unit generates vibrations in the ultrasonic frequency range in an ultrasonic wave band, the vibration generation unit includes an ultrasonic transducer.

The cell detachment system 1100 according to the present exemplary embodiment may include the information processing device 1000 according to the present exemplary embodiment described above, and a measurement device 1010 that performs a measurement to acquire information about the cells. The measurement device 1010 is not particularly limited as long as the measurement device 1010 can acquire information about the cells. The measurement device 1010 may include an image capturing device for acquiring an image of cells or a moving image of cells, and an analysis unit for analyzing the image of cells or the moving image of the cells obtained by the image capturing device, thus acquiring the information about the cells.

### (Hardware Configuration of Information Processing Device)

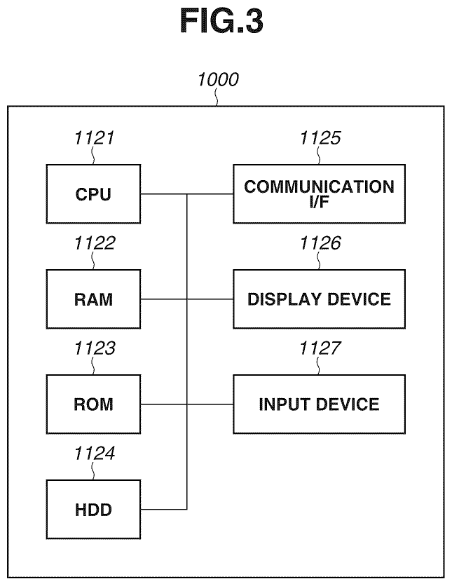

FIG. 3 is a block diagram illustrating a hardware configuration example of the information processing device 1000 according to the present exemplary embodiment. The information processing device 1000 includes a central processing unit (CPU) 1121, a random access memory (RAM) 1122, a read-only memory (ROM) 1123, a hard disk drive (HDD) 1124, a communication interface (I/F) 1125, a display device 1126, and an input device 1127. These units are interconnected via a bus or the like.

The CPU 1121 is a processor that reads out programs stored in the ROM 1123 or the HDD 1124 into the RAM 1122, and executes the programs to perform arithmetic processing, control processing, and the like on each unit of the information processing device 1000. The processing to be performed by the CPU 1121 includes acquisition of the information about the cells, acquisition of the information about the detachment conditions, and acquisition of the information about culture conditions.

The RAM 1122 is a volatile storage medium and functions as a working memory for the CPU 1121 to execute programs. The ROM 1123 is a non-volatile storage medium and stores firmware and the like to be used for operation of the information processing device 1000. The HDD 1124 is a non-volatile storage medium and stores the information about the cells, the information about the detachment conditions, and the information about culture conditions in association with each other.

The communication I/F 1125 is a communication device based on standards such as Wireless Fidelity (Wi-Fi®), Ethernet, or Bluetooth®. The communication I/F 1125 is used for communication with the measurement device 1010 and the detachment device 1011, which are described below, other computers, and the like.

The input device 1127 is a device for inputting information to the information processing device 1000. Typically, the input device 1127 is a user interface for a user to operate the information processing device 1000. Examples of the input device 1127 include a keyboard, buttons, a mouse, and a touch panel.

The display device 1126 is a device for the information processing device 1000 to output information to an external device. Typically, the display device 1126 is a user interface for presenting information to the user. Examples of the display device 1126 include a display and a speaker.

The above-described configuration of the information processing device 1000 is merely an example and can be modified as appropriate. Examples of a processor to be mounted on the information processing device 1000 include not only the CPU 1121 described above, but also a graphics processing unit (GPU), an application-specific integrated circuit (ASIC), and a field-programmable gate array (FPGA). A plurality of such processors may be provided and the plurality of processors may perform processing in a distributed manner. A function for storing information such as image data in the HDD 1124 may be provided in another data server, instead of providing the function in the information processing device 1000. The HDD 1124 may be a storage medium such as an optical disk, a magneto optical disk, or a solid state drive (SSD).

The CPU 1121 executes programs to perform predetermined arithmetic processing. The CPU 1121 executes programs to thereby control each unit in the information processing device 1000. Such processing enables the CPU 1121 to implement the functions of the cell information acquisition unit 1001 and the detachment condition acquisition unit 1002.

The hardware configuration illustrated in FIG. 3 is merely an example, and any other devices may be added, or some of the devices may be omitted, as long as the functions of the information processing device 1000 according to the present exemplary embodiment can be implemented. Some of the devices may be replaced with other devices including similar functions. Further, some of the functions may be provided by other devices via a network, and the functions constituting the present exemplary embodiment may be implemented by distributing the functions to a plurality of devices. For example, the HDD 1124 may be replaced with an SSD using a semiconductor element such as a flash memory.

### (Cells)

In the present exemplary embodiment, the cell type is not particularly limited as long as the cells can be cultured in vitro and adhere to a culture material. Examples of the cells include not only various culture cells lines such as Chinese hamster ovary (CHO) cells, mouse connective tissue L929 cells, mouse skeletal muscle myoblast cells (C2C12 cells), normal diploid fibroblasts derived from human fetal lung (TIG-3 cells), human embryonic kidney-derived cells (HEK293 cells), human alveolar basal epithelial adenocarcinoma cells (A549 cells), murine macrophage-like cells (RAW 264.7), and human cervical cancer-derived Hela cells. In addition, epithelial and endothelial cells from various tissues and organs in vivo, contractile cells such as skeletal muscle cells, smooth muscle cells, and cardiomyocytes, as well as neurons and glial cells of the nervous system, fibroblasts, hepatocytes involved in the metabolism of the living body, adipocytes, and various stem cells with differentiation potential-such as induced pluripotent stem (iPS) cells, embryonic stem(ES) cells, embryonic germ (EG) cells, embryonal carcinoma (EC) cells, mesenchymal stem cells, hepatic stem cells, pancreatic stem cells, skin stem cells, muscle stem cells, and germline stem cells-as well as progenitor cells of each tissue and cells induced to differentiate from them, may also be used.

In the present exemplary embodiment, the term “cell” may refer to individual cells (commonly referred to as single cells), or to sheet-like cell cultures (cells sheets). Among these examples, the cell detachment method according to the present embodiment is particularly suitable for cells with strong intercellular adhesion, cells with high adhesion strength to the substrate, and cells with high trypsin sensitivity. To satisfy the demand for large-scale cell culture, the method is suitable for, in particular, for example, CHO cells for use in protein production and mesenchymal stem cells applicable to cell therapy.

### (Culture Substrate)

In the present exemplary embodiment, the term “substrate” refers to a culture substrate for use in cell culture. The culture substrate is not particularly limited as long as the culture substrate has cellular adhesiveness. Examples of the culture substrate include a flask, a tissue culture flask, a dish, a petri dish, a tissue culture dish, a multiple dish, a microplate, a multi-well plate, a multiple plate, a culture bag, and a bottle.

In the present exemplary embodiment, the material for the culture substrate may be any material that is chemically stable and capable of culturing the desired cells. Examples of the material include polyethylene, polypropylene, polycarbonate, polystyrene, polyvinyl chloride, nylon, polyurethane, polyurea, polylactate, polyglycolic acid, polyvinyl alcohol, polyvinyl acetate, poly (meth) acrylate, poly (meth) acrylic acid derivatives, polyacrylonitrile, poly (meth) acrylamides, poly (meth) acrylamide derivatives, polysulfone, cellulose, cellulose derivatives, polysilicone, polymethylpentene, glass, and metal. Among these examples, polystyrene can be used.

### Second Exemplary Embodiment

### Information Processing Method

An information processing method according to a second exemplary embodiment of the present disclosure includes at least one of the following steps:
(1) a cell information acquisition step of acquiring information about cells (step S1001);
(2) a detachment condition acquisition step of acquiring information about detachment conditions for detaching the cells adhering to a culture substrate from the culture substrate by applying an external force to the culture substrate (step S1002); and
(3) a storage step of storing the information about the cells and the information about the detachment conditions in association with each other (step S1003).

The information processing method according to the present exemplary embodiment may also include acquiring information about culture conditions for the cells. In such a case, in step (3), the information about the cells, the information about the detachment conditions, and the information about culture conditions for the cells may be stored in association with one another.

### Third Exemplary Embodiment

### Graphical User Interface (GUI)

In the case of detaching cells with a plurality of detachment methods, the configuration, loading, and saving of detachment conditions require different considerations from those used in a GUI designed for the user to control a detachment method using a single detachment unit. For example, a GUI having the following configuration may be used.

A driving state and a non-driving state of the detachment unit (detachment device) that operates in a plurality of modes over time in each mode may be displayed in association with driving conditions input by the user on the GUI. The driving and non-driving states in each mode may be displayed as a bar chart arranged in chronological order, with the length of each bar proportional to the duration of the corresponding state. This bar chart may be displayed on a common time axis in a vertical, horizontal, or concentric circular layout on the GUI. The shape of the bar chart may be varied according to numerical values input by the user via the GUI. On the GUI, a display portion corresponding to a specific mode on the bar chart may be highlighted with a color during the operation in the specific mode in accordance with the passage of time. The bar chart may include an indicator indicating the current time, and the position of the indicator may be changed on the bar chart in accordance with the passage of time. A storage medium for caching or store detachment conditions may be provided. The GUI may include a continuation check box. If a detachment operation by cell detachment device is started with the continuation check box selected, conditions of the detachment operation may be added to the existing cache. Conversely, if the detachment operation is started with the continuation check box unselected, the existing cache may be overwritten with the new detachment conditions. During storage of the detachment conditions upon completion of the detachment operation, the cache is stored as experimental data in a format that allows writing in and reading from the storage medium, and the cache at that time may be deleted from the storage medium. Furthermore, in a case where the detachment operation ends while the continuation check box remains selected, a cell sheet adhesion prevention operation may be performed until the next detachment operation begins, unless the check box is manually deselected. The adhesion prevention operation may be a detachment operation in one of the plurality of modes, or in a combination of some of the modes. The cell detachment device may include a storage medium for storing the detachment conditions, and the GUI may include a plurality of detachment condition edit buttons for writing or reading the detachment conditions to or from the storage medium. Among the plurality of detachment condition edit buttons, the one corresponding to the detachment condition selected by the user and under which the detachment operation is started may be located on the GUI in the order of execution of the detachment operation. The cell detachment device may include a storage medium for storing the detachment conditions, and the GUI may include a detachment condition reading unit for reading a detachment condition file from the storage medium. When the detachment operation is actually executed based on the detachment condition file selected by the detachment condition reading unit, an edit operation may be performed on the detachment condition file. The edit operation may be alteration of a time stamp of the detachment condition file. The edit operation may be addition of a time stamp to a prefix of a file name of the detachment condition file. The edit operation may be alteration of the detachment condition file within a range in which settings of a setting file are not changed.

Examples of the GUI used in detaching cells by a plurality of detachment methods as described above will be described below.

### EXAMPLE OF CELL DETACHMENT SYSTEM

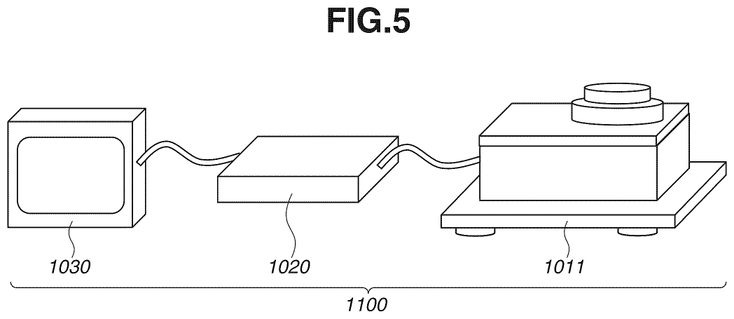

FIG. 5 is an overview diagram of the cell detachment system 1100 according to the present exemplary embodiment. The detachment device 1011 detaches the cells adhering to the culture substrate from the culture substrate by applying an external force to the culture substrate. In the detachment device 1011, electric power supplied from a control unit 1020 is converted into a mechanical motion for application of an external force by a direct current (DC) electric motor or the like. The control unit 1020 generates an electric signal based on designated detachment conditions and transmits the electric signal to the detachment device 1011. The reception unit 1003 (not illustrated) receives the detachment conditions designated by the user via the GUI and transmits a signal indicating the designated detachment conditions to the control unit 1020 to thereby control the start and stop of the detachment operation. The cell detachment system 1100 also includes the storage unit 1005 (not illustrated) for storing the detachment conditions.

The storage unit 1005 is configured to temporarily store the detachment conditions as a cache. A display unit 1030 is configured to display the detachment conditions under the control of the display control unit 1004.

### EXAMPLE 1 OF GUI

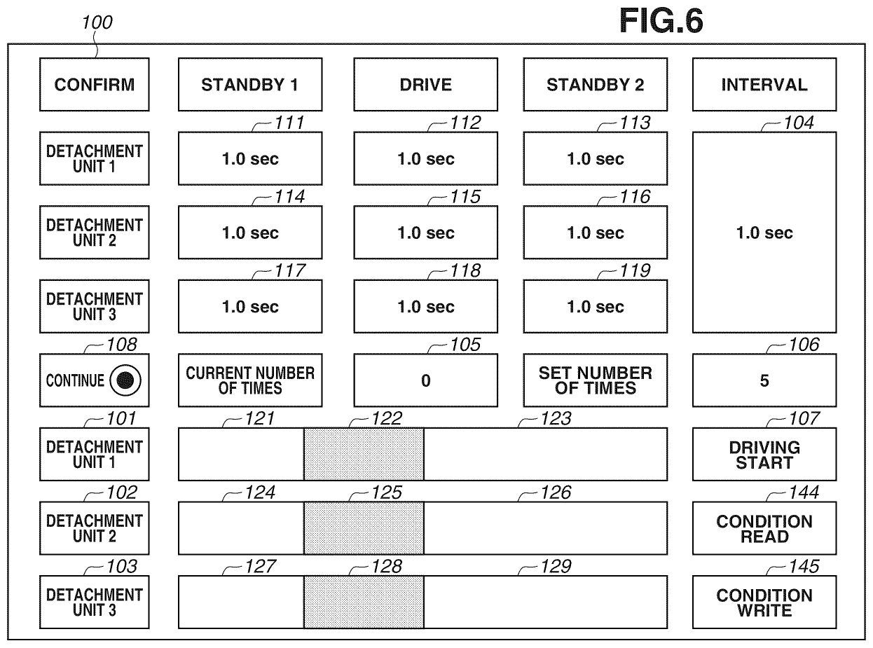

FIG. 6 illustrates an operation screen of a GUI according to Example 1. This operation screen is displayed on the display unit 1030. As described above, in this example, the detachment operation includes three modes, namely, a detachment unit 1, a detachment unit 2, and a detachment unit 3. The operation screen includes buttons 101, 102, and 103 for setting detachment conditions for the detachment unit 1, the detachment unit 2, and the detachment unit 3. An example of such detachment units is a unit for executing the method described above in regard to the application of an external force. When any one of the buttons 101, 102, and 103 is pressed, a detachment condition setting wizard is displayed, so that the user can set detachment conditions. In this example, the detachment operation in the three modes is performed repeatedly at intervals and for a number of repetitions designated by the user. The user can designate the number of repetitions and input the designated number of repetitions to a set-number-of-times input section 106. After a driving operation is started, the current number of repetitions is displayed as “current number of times” on the display unit 105. In each of the three modes, “standby 1”, “drive”, “standby 2”, “interval”, and “transition of driving states” are repeated a set number of times. The term “drive” refers to a state where the detachment operation is actually being performed in the corresponding mode. In the other driving states, the detachment operation is suspended. The user can input the duration of each driving state to the corresponding state duration input sections 111 to 119. The user can input the duration of each of “standby “1”, “drive”, and “standby 2” states to the state duration input sections 111 to 113, respectively, in a vibration mode. Similarly, the user can input the duration of each state in the mode of the detachment unit 2 to the corresponding state duration input sections 114 to 116, and also can input the duration of each state in the mode of the detachment unit 3 to the state duration input sections 117 to 119. The duration of the interval state is common across the three modes and is input to an interval time input section 104. The drive cycle is the sum of the durations of “standby 1”, “drive”, “standby 2”, and “interval” states. The three modes may have different drive cycles depending on values input by the user. In such cases, if a confirm button 100 is pressed after values are input, the duration of “standby 2” is adjusted to automatically correct input values for “standby 2” in the three modes so that the cycle durations become uniform across all three modes. State transition display bars 121 to 129 are provided to indicate an operation state and a non-operation state in the three modes. Only a time region in the driving state during one cycle is displayed by dots. In FIG. 6, the display of each state duration in the three modes does not correspond to the display of each state transition display bar.

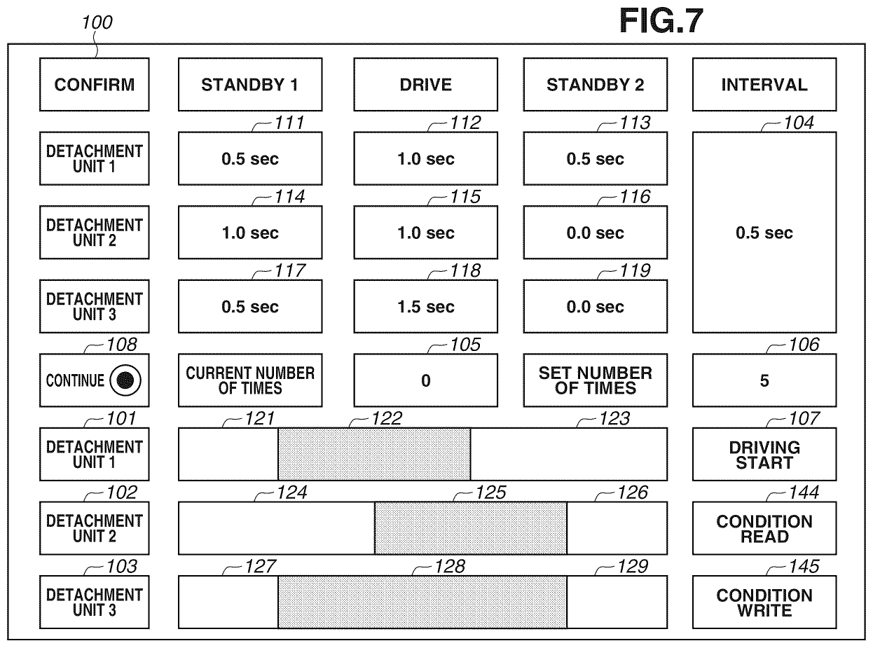

FIG. 7 illustrates a GUI operation screen according to an exemplary embodiment of the present disclosure, which is displayed after each state duration is confirmed. FIG. 7 illustrates a state where the state durations of the state duration input sections 111 to 119 and the interval time are input on the operation screen illustrated in FIG. 6, and then the confirm button 100 is pressed. In FIG. 7, the cycle of the vibration mode includes durations of 0.5 seconds for the “standby 1” state, 1.0 second for the “drive” state, 0.5 seconds for the “standby 2” state, and 0.5 seconds for the “interval” state, resulting in a total operation cycle of 0.5+1.0+0.5+0.5=2.5 seconds. A portion corresponding to the duration of the “standby 1” state is displayed as the state transition display bar 121 corresponding to the vibration mode. A portion corresponding to the duration of the “drive” state is displayed as the state transition display bar 122 corresponding to the vibration mode. A portion corresponding to the durations of the “standby 2” and “interval” states is displayed as the state transition display bar 123 corresponding to the vibration mode. The length of each state transition display bar is displayed in proportion to the corresponding duration. A similar configuration is applied to the mode of the detachment unit 2 and the mode of the detachment unit 3. As illustrated in FIG. 7, when the confirm button 100 is pressed and the duration is confirmed, the state transition display bars 121 to 129 are displayed in such a manner that the state transition display bars 121 to 129 correspond to the values input to the state duration input sections 111 to 119 and the interval time input section 104. This configuration enables the user to visually check the conditions numerically input, thereby making it possible to prevent erroneous input of the detachment conditions. Particularly, in a cell detachment system using three modes, it is important to determine which modes are to be used in combination for action. When the state transition display bars are vertically arranged as illustrated in FIGS. 6 and 7, a starting point and an end point of the driving state in each mode can be easily compared. Accordingly, this configuration is effective when detachment conditions are considered.

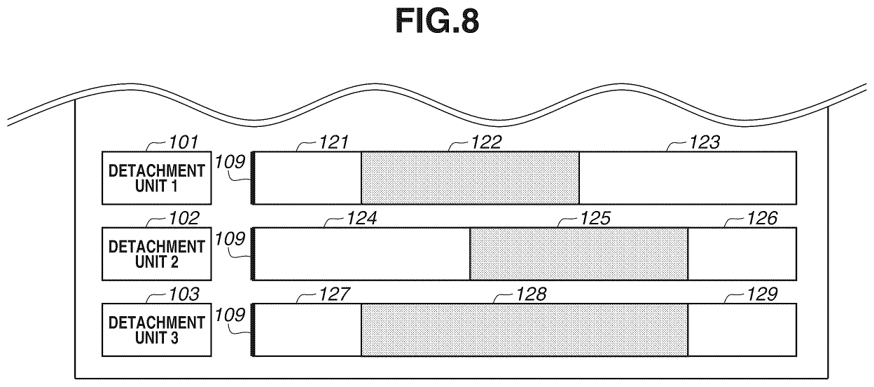

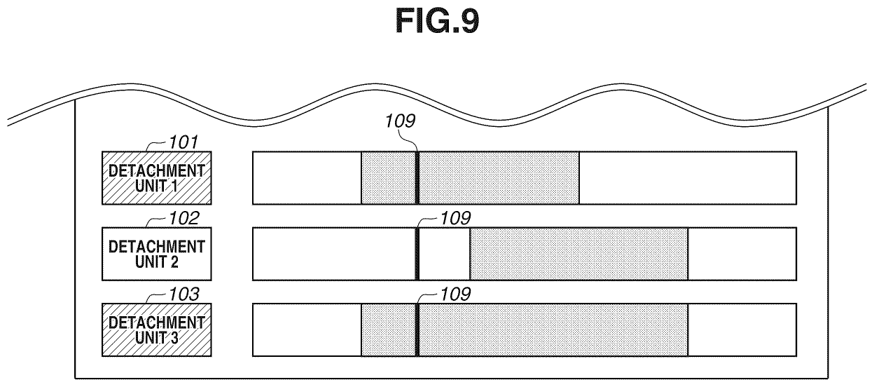

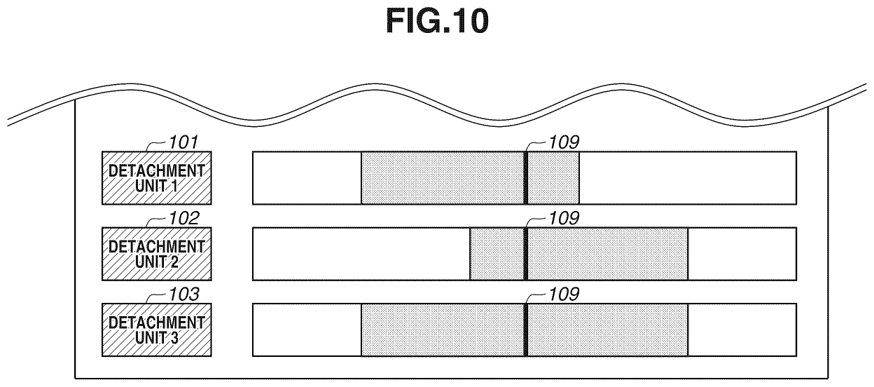

FIGS. 8 to 12 each illustrate a time change of an indicator position on each state display bar according to this example. When a driving start button 107 illustrated in FIG. 7 is pressed and the detachment operation is started, an indicator 109 is displayed on each of the state transition display bars 121 to 129. As time progresses, the indicator 109 moves from left to right on each state transition display bar. FIG. 8 illustrates the position of the indicator 109 immediately after the driving start button 107 is pressed, and the position corresponds to the leftmost position on each state transition display bar. As time progresses, the indicator 109 in each mode moves rightward on the state transition display bar corresponding to the “standby 1” state. FIG. 9 illustrates the position of the indicator 109 when 0.75 seconds have elapsed after the detachment operation is started. At this time point, the driving state is set in the vibration mode and a tapping mode, so that a vibration condition setting button 101 and a tapping condition setting button 103 are highlighted. FIG. 10 illustrates the position of the indicator 109 when 1.25 seconds have elapsed after the detachment operation is started.

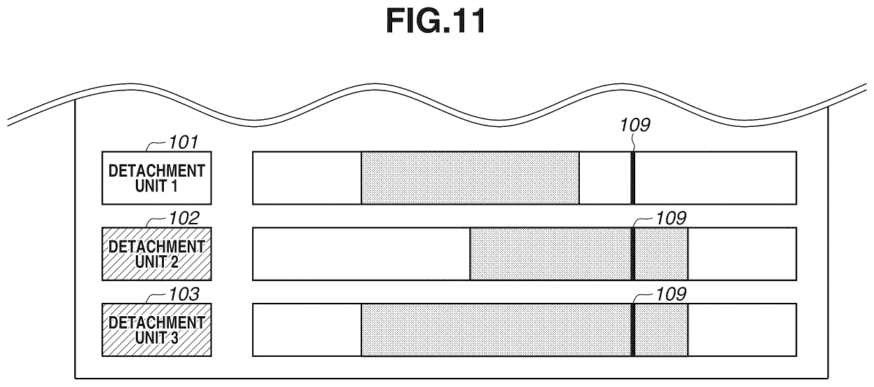

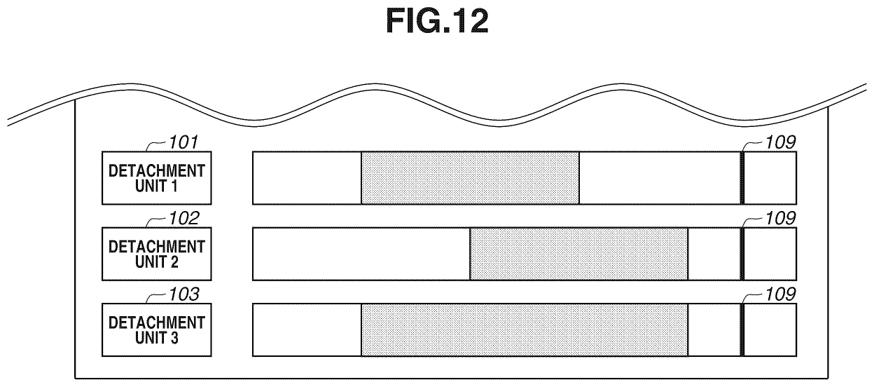

At this time point, the driving state is set in all the modes, so that the vibration condition setting button 101, a shaking condition setting button 102, and the tapping condition setting button 103 are highlighted. FIG. 11 illustrates the position of the indicator 109 when 1.75 seconds have elapsed after the detachment operation is started. At this time, the driving state is set in a shaking mode and the tapping mode, and thus the shaking condition setting button 102 corresponding to the detachment unit 2 and the tapping condition setting button 103 corresponding to the detachment unit 3 are highlighted. FIG. 12 illustrates the position of the indicator 109 when 2.25 seconds have elapsed after the detachment operation is started. At this time, the non-driving state is set in all the modes, and thus there are no condition setting buttons highlighted.

As described above, the indicator 109 moves on each of the state transition display bars 121 to 129 as time progresses. Further, when the indicator 109 is located on the position corresponding to the driving state in each mode, the condition setting button in each mode is highlighted. This configuration enables the user to easily recognize the driving state or the non-driving state at the present time.

### EXAMPLE 2 OF GUI

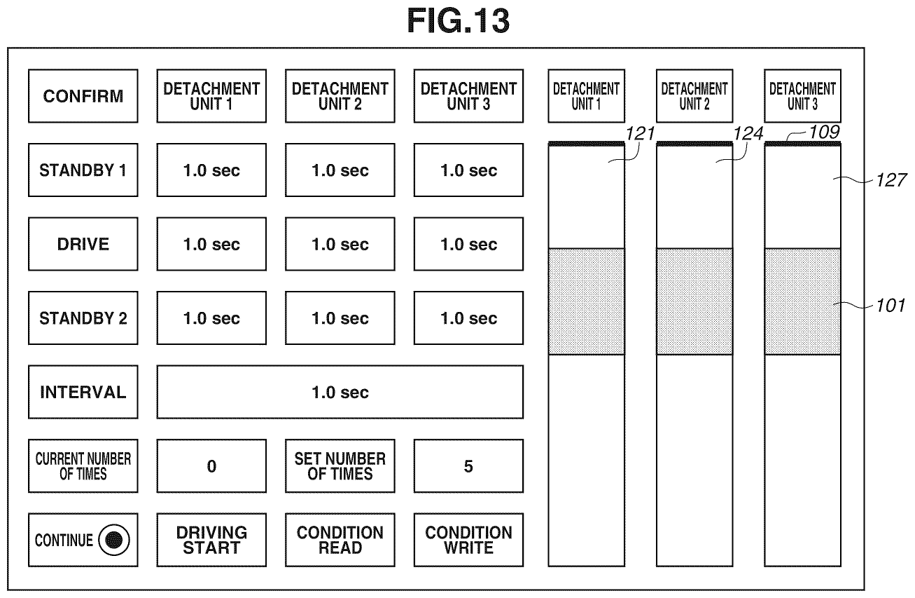

A configuration of a cell detachment system according to Example 2 is similar to that of Example 1. Only the configuration of the display screen of the GUI on the display unit 1030 is different from that of Example 1. FIG. 13 illustrates an operation screen of the GUI according to Example 2. Unlike the GUI operation screen according to Example 1 illustrated in FIG. 6, the state transition display bars are displayed in the longitudinal direction. As in Example 1, this system also facilitates the user to visually recognize the driving state or the non-driving state for each mode.

### EXAMPLE 3 OF GUI

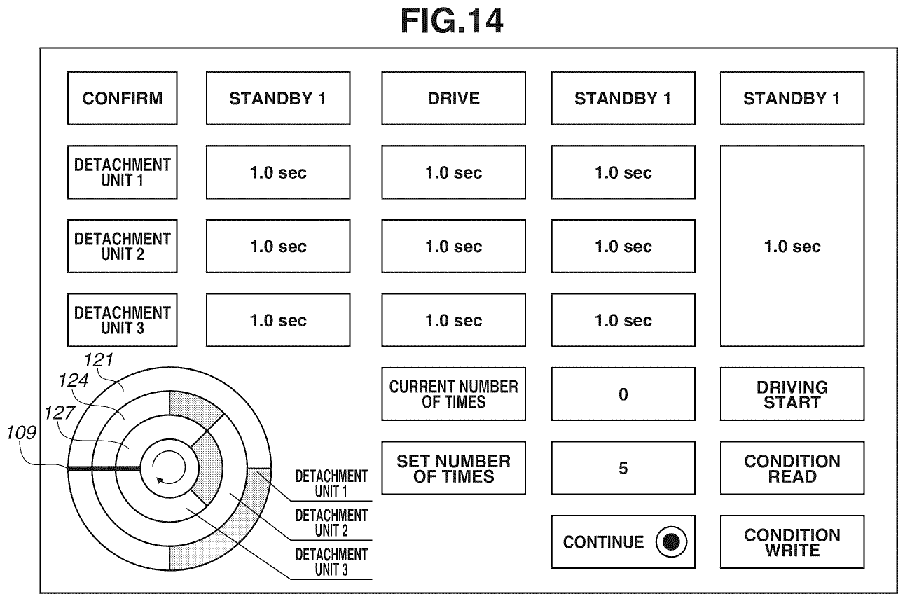

A configuration of a cell detachment system according to Example 3 is similar to that of Example 1. Only the configuration of the display screen of the GUI on the display unit 1030 is different from that of Example 1. FIG. 14 illustrates an operation screen of the GUI according to Example 3. Unlike the GUI operation screen according to Example 1 illustrated in FIG. 6, the state transition display bars are concentrically displayed. As in Example 1, this system also facilitates the user to visually recognize the driving state or the non-driving state for each mode.

### EXAMPLE 4 OF GUI

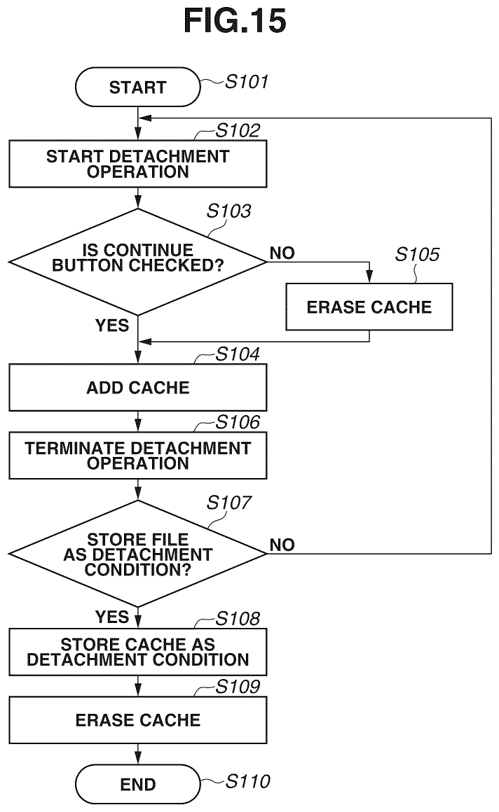

A configuration of a cell detachment system according to Example 4 is similar to that of Example 1. The configuration of the display screen of the GUI on the display unit 1030 is also similar to that of Example 1 as illustrated in FIG. 6. In the example illustrated in FIG. 6, when a detachment condition write button 145 is pressed, the detachment conditions can be stored. In Example 4, this detachment condition storage sequence is changed depending on whether a continue button 108 is checked. FIG. 15 is a flowchart illustrating a detachment condition storage operation according to Example 4. After the detachment conditions is input, in step S102, the driving start button 107 is pressed to start the detachment operation. In step S103, it is determined whether the continue button 108 is selected. If it is determined that the continue button 108 is selected (YES in step S103), the processing proceeds to step S104. In step S104, information about the detachment conditions set in the current detachment operation is added to the cache in a state where the cache of information about the detachment conditions used for the previous detachment operation is maintained. If it is determined that the continue button 108 is unselected (NO in step S103), the processing proceeds to step S105. In step S105, the cache of information about the detachment conditions used for the previous detachment operation is deleted and information about the detachment conditions set in the current detachment operation is stored as a cache. In step S106, the detachment operation is terminated. In step S107, it is determined whether the detachment condition write button 145 is pressed to store the detachment conditions. If it is determined that the detachment conditions is to be stored (YES in step S107), the processing proceeds to step S108. In step S108, the cache in the storage device is stored as a detachment condition file. After the file is stored, in step S109, the cache is erased. Then, the processing proceeds to step S102 to transition to the next detachment operation without storing the detachment conditions.

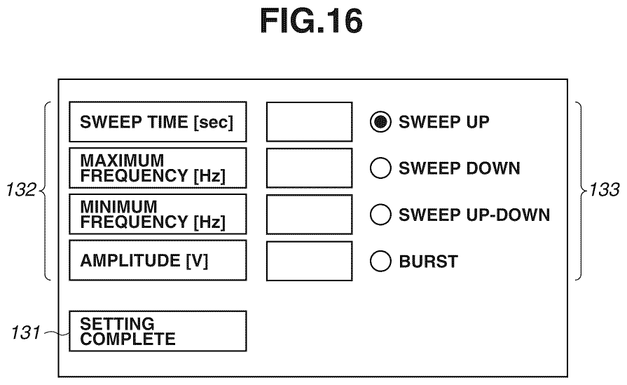

FIG. 16 illustrates a vibration condition setting screen according to Example 4. When the vibration condition setting button 101 is pressed, the screen can be displayed. In the vibration mode, a designated sinusoidal voltage signal having a designated amplitude for a designated sweep time in a designated frequency range is input to an ultrasonic wave generation section 303. Thus, a sweep time, a maximum frequency, a minimum frequency, and a (sine-wave) amplitude can be designated in a detachment conditions input unit in the vibration mode. In a detachment pattern selection section 133, the following sweep patterns can be selected: (i) a sweep up pattern in which a sweep frequency sweeps from a minimum frequency to a maximum frequency as a sweep direction; (ii) a sweep down pattern of sweeping from the maximum frequency to the minimum frequency; (iii) a sweep up-down pattern of reciprocating between the minimum frequency and the maximum frequency once within the sweep time; and (iv) a burst pattern with a constant frequency.

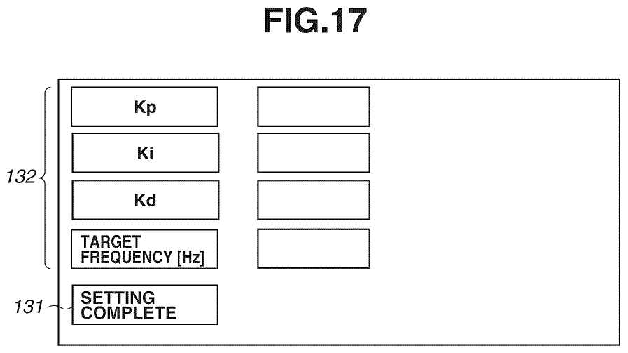

FIG. 17 illustrates a condition setting screen for the detachment unit 2 according to Example 4. If shaking is set as the detachment unit 2, a target frequency for setting the frequency of the reciprocating operation and proportional-integral-derivative (PID) coefficients (Kp, Ki, Kd) for controlling a direct-acting electric motor that causes the detachment unit 2 to operate can be designated.

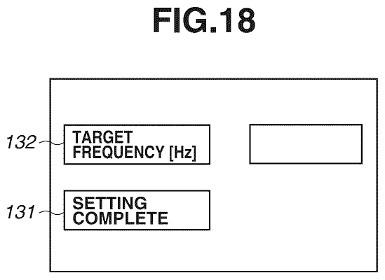

FIG. 18 illustrates a condition setting screen for the detachment unit 3 according to Example 4. If tapping is set as the detachment unit 3, a frequency for tapping a culture substrate (not illustrated) can be designated.

A configuration in which conditions other than the detachment conditions illustrated in FIGS. 16 to 18 can be set may be employed. An example of tapping conditions is an impact force of tapping on a culture substrate (culture vessel, not illustrated) during tapping. Other examples of experimental conditions that can be designated include a date of experiment, a location, an operation company name, the cell type, a lot number, a passage number, the number of days of culturing, the number of cells, the number of cells to be seeded, the type of a medium, the volume of culture medium, additives to the culture medium, a culture substrate (culture vessel) cleaning method, the type of washing solution, the volume of washing solution, the number of washing operations, the type of detachment solution for detaching a cell sheet, the type and volume of the detachment solution, such as ethylenediaminetetraacetic acid (EDTA), phosphate buffered saline (PBS), or trypsin, a survival rate after detachment, the type of a culture substrate (culture vessel), the type of a vibration transmitter, temperature and humidity as experimental environments, and the level of carbon dioxide (CO₂).

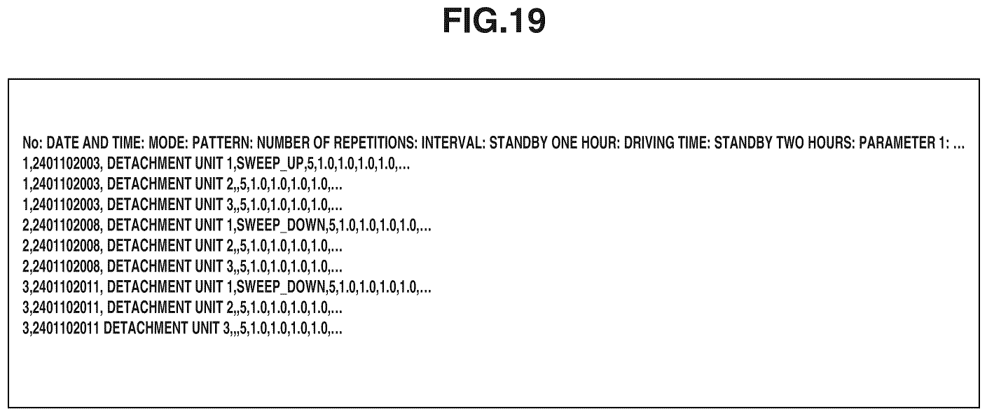

FIG. 19 illustrates an example of the content of the detachment condition file in Example 4. This file corresponds to a case where the detachment operation has been performed three times in a state where the continue button 108 is pressed, and the detachment condition storage operation has been skipped in the first and second detachment operations. It can be seen that the detachment conditions for the first to third detachment operations are arranged in chronological order and stored as one file. For each detachment operation, one line is allocated for each of the three delamination methods. The order of the recorded conditions is as follows: lot number, date and time, detachment method, and, if the vibration mode is used, number of repetitions, interval, duration of “standby 1” state, duration of “drive” state, duration of “standby 2” state, followed by detachment conditions specific to each method.

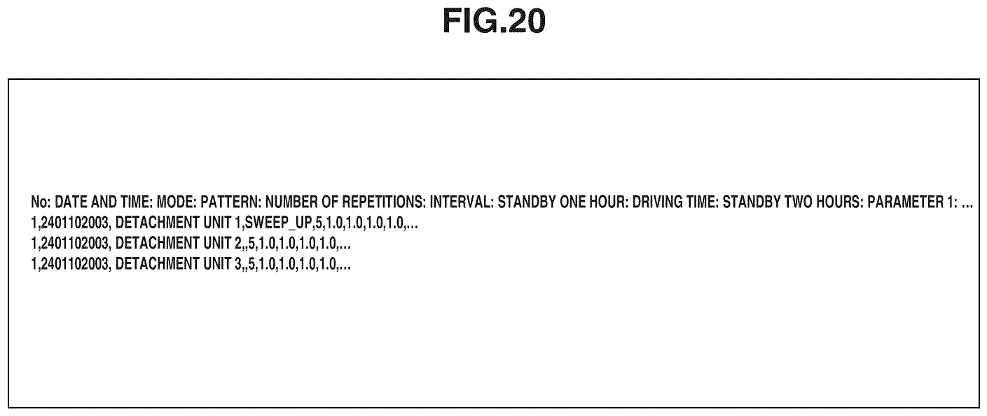

FIG. 20 illustrates an example of the content of the detachment condition stored file in Example 4. This file corresponds to a case where the detachment operation is performed twice in a state where the continue button 108 is not pressed, and the detachment condition storage operation is skipped in the first detachment operation. The detachment conditions for the first detachment operation are not recorded in the stored file and only the detachment conditions for the second detachment operation are recorded in the file.

The provision of the continue button 108 as described above eliminates the need for storing the experimental conditions for each operation and makes it possible to collectively store the detachment conditions as one file after any number of detachment operations are finished.

### EXAMPLE 5 OF GUI

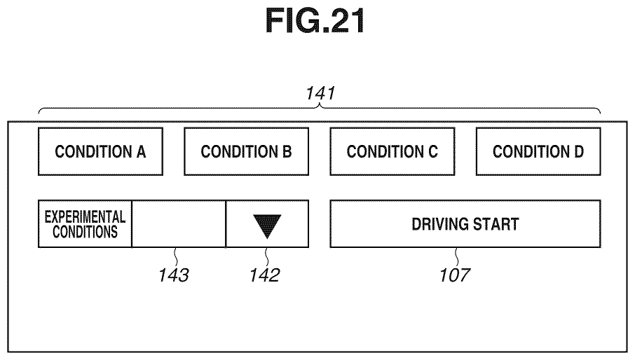

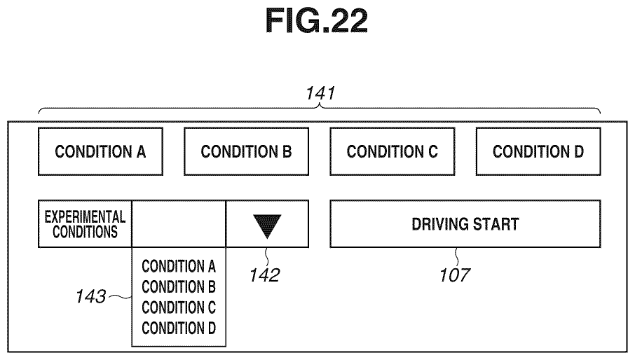

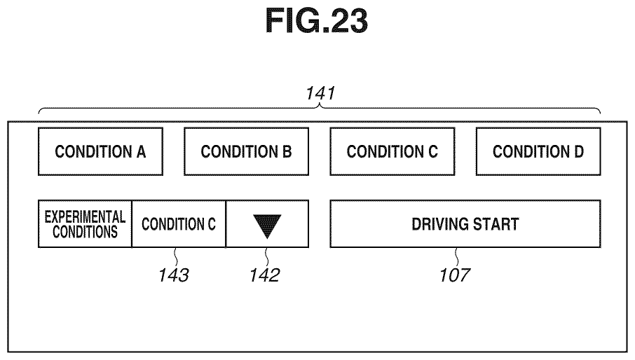

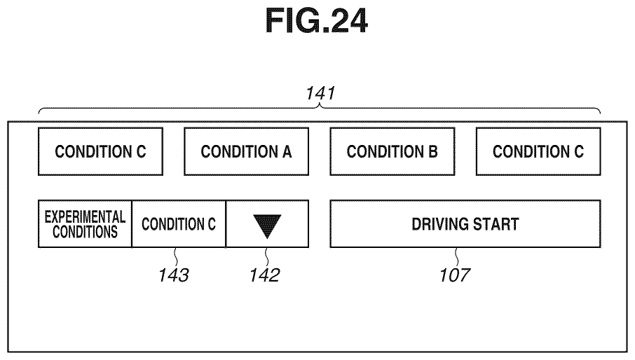

FIGS. 21 to 24 each illustrate a detachment condition setting screen in Example 5. A configuration of a cell detachment system according to Example 5 is similar to that of Example 1. Only the configuration of the display screen of the GUI on the display unit 1030 is different from that of Example 1. In this example, four detachment condition edit buttons 141 are arranged as illustrated in FIG. 21. When any one of the detachment condition edit buttons 141 is pressed, a screen for editing the experimental conditions is started as in Example 4. The detachment condition edit buttons 141 are arranged in order from left to right in chronological order of detachment operations actually executed based on the detachment conditions. For example, in a display state illustrated in FIG. 21, the detachment conditions used are displayed in the order of A→B→C→D. Here, when a detachment condition selection tab 142 is pressed, callable detachment conditions are displayed as illustrated in FIG. 22 and one of the detachment conditions is selected. FIG. 23 illustrates a state where the condition Cis selected. In this case, when the driving start button 107 is pressed to start the detachment operation, the detachment conditions used in the preceding operations are displayed as the condition C, the condition A, the condition B, and the condition C in chronological order. Accordingly, the detachment condition edit buttons 141 are arranged as illustrated in FIG. 24. This allows users to easily recall the detachment conditions used in chronological order, enabling GUI operations with improved convenience in setting conditions.

### EXAMPLES 6 AND 7 OF GUI

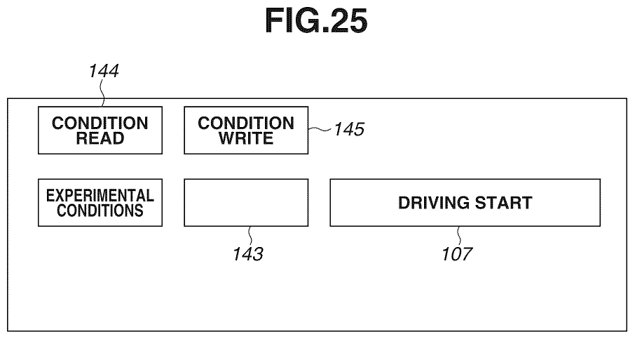

FIG. 25 illustrates a detachment condition setting screen in Examples 6 and 7. A configuration of the respective cell detachment systems according to Example 6 and Example 7 is similar to that of Example 1. Only the configuration of the display screen of the GUI on the display unit 1030 is different from that of Example 1. A detachment condition read button 144 is displayed on the GUI operation screen. When the detachment condition read button 144 is pressed, a screen for selecting a detachment condition file stored in the storage device can be called.

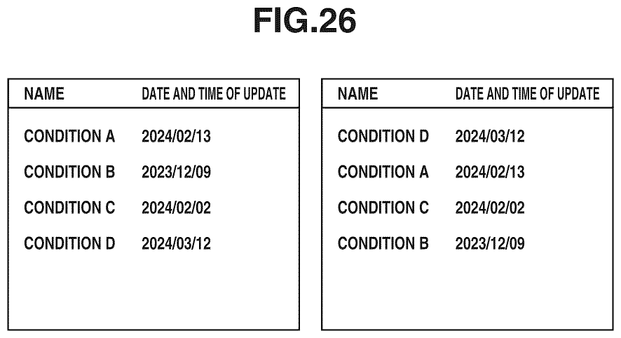

The selected detachment condition is displayed on a detachment condition display section 143. When the driving start button 107 is pressed, the detachment operation is started based on the detachment condition displayed on the detachment condition display section 143. FIG. 26 illustrates a wizard screen to be displayed upon selection of a detachment condition file according to Example 6. This wizard screen is started when the detachment condition read button 144 is pressed. In Example 6, when the detachment operation is actually started based on the selected detachment condition file, the time stamp of the detachment condition file is overwritten with the time of execution. For example, assume that the name and time stamp of the detachment condition file on a detachment condition selection wizard screen are displayed as a left screen illustrated in FIG. 26 before the detachment operation is started. In this case, when the file for the condition D is selected as the detachment conditions and the detachment operation is started, the time stamp of the detachment condition file for the condition D is overwritten with “2024 Mar. 12” as the date and time when the detachment operation is started. This configuration makes it possible to arrange the detachment condition files in order of date and time of update (time stamp) on a detachment condition selection wizard screen displayed as a right screen illustrated in FIG. 26. In other words, the detachment condition files can be arranged on the wizard screen in reverse chronological order of use time.

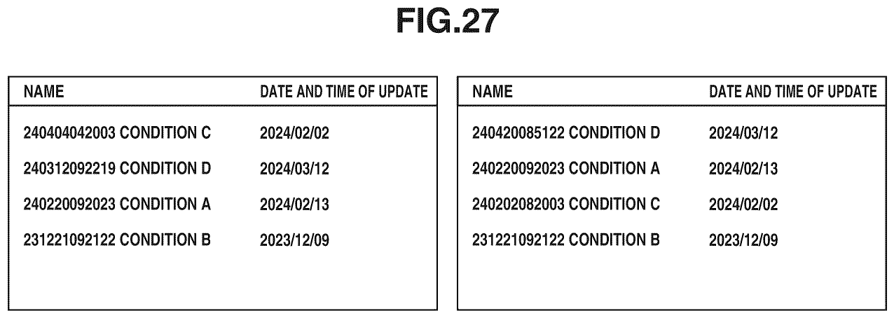

FIG. 27 illustrates a wizard screen to be displayed upon selection of a detachment condition file according to Example 7. In Example 7, when the detachment operation is actually started based on the selected detachment condition file, the time stamp of the detachment condition file is recorded in the prefix of the file name. For example, assume that the name and time stamp of the detachment condition file on the detachment condition selection wizard screen are displayed as a left screen illustrated in FIG. 27 before the detachment operation is started. In this case, when the file for the condition D is selected as the detachment conditions and the detachment operation is started, the prefix “240312092219” of the detachment condition file for the condition D is overwritten with “240420085122” as the date and time when the detachment operation is started. With this configuration, the detachment condition files are sorted by name on a detachment condition selection wizard screen displayed as a right screen illustrated in FIG. 27, thereby making it possible to arrange the detachment condition files in chronological order of detachment conditions actually used.

As described above, the GUI described in the present exemplary embodiment provides high user operability in detaching cells within a culture vessel in a plurality of modes, during setting of detachment conditions, reading of detachment conditions, storage of detachment conditions, and the detachment operation to be actually performed based on the detachment conditions. Specifically, in the case of detaching a cell sheet within a culture vessel in a plurality of modes, detachment condition settings can be checked at any time by visualizing the detachment condition settings. Further, the detachment conditions can be sequentially called in chronological order of detachment operations during reading of detachment conditions, thereby enhancing user convenience. Furthermore, to reduce the operational burden, the experimental conditions from a plurality of detachment operations leading up to detachment can be consolidated into a single detachment condition file in storing. Thus, this enables the provision of a highly operable GUI for users performing cell detachment operations.”

For example, the present disclosure may also be implemented as follows. That is, an information processing program for implementing one or more functions according to the exemplary embodiments described above is supplied to a system or an apparatus via a network or a storage medium. One or more processors in a computer of the system or the apparatus read out and execute the information processing program. The present disclosure may also be implemented by, for example, a circuit (e.g., an ASIC) for implementing one or more functions according to the exemplary embodiments.

While exemplary embodiments of the present disclosure have been described above, the exemplary embodiments are merely specific examples for carrying out the present disclosure. The technical scope of the present disclosure should not be interpreted in a limited way. That is, the present disclosure can be carried out in various forms without departing from the technical idea or the main features thereof. For example, it should be understood that an example where a part of the configuration according to any of the exemplary embodiments is added to any of the other exemplary embodiments, and an example where a part of the configuration according to any of the exemplary embodiments is replaced with a part of the configuration according to any of the other exemplary embodiments are also included in the exemplary embodiments of the present disclosure.

Exemplary embodiments of the present disclosure includes the following methods and configurations.

### (Configuration 1)

An information processing device, comprising:
cell information acquisition unit configured to acquire information about a cells;
a detachment condition acquisition unit configured to acquire information about a detachment condition for detaching the cell adhering to a culture substrate from the culture substrate by an external force being applied to the culture substrate; and
a storage unit configured to store the information about the cell and the information about the detachment condition in association with each other.

### (Configuration 2)

The information processing device according to configuration 1, wherein vibration is applied to the culture substrate to apply the external force to the culture substrate.

### (Configuration 3)

The information processing device according to configuration 2, wherein the information about the detachment condition includes at least one piece of information selected from the group consisting of information about an intensity of the vibration to be applied to the culture substrate, information about a frequency of the vibration to be applied to the culture substrate, and information about a duration of vibration to be applied to the culture substrate.
(Configuration 4)

The information processing device according to configuration 2,
wherein the vibration to be applied to the culture substrate is generated by application of an ultrasonic wave generated by an ultrasonic transducer, and
wherein information about an intensity of the vibration to be applied to the culture substrate includes information about a voltage to be applied to the ultrasonic transducer.

### (Configuration 5)

The information processing device according to any one of configurations 1 to 4, wherein the information about the detachment condition includes at least one piece of information selected from the group consisting of information about detachment solution to be used to detach the cell from the culture substrate, and information about an acoustic matching material.

### (Configuration 6)

The information processing device according to any one of configurations 1 to 5,
wherein the information about the detachment condition is information obtained based on information about a detachment result of detaching the cell from the culture substrate, and
wherein the information about the detachment result includes at least one piece of information selected from the group consisting of information about a detachment rate of the cell, information about a survival rate of the detached cell, information about a growth rate of the detached cell, and information about functionality of the detached cell.

### (Configuration 7)

The information processing device any one of configurations 1 to 6, wherein the information about the cell includes information about cell type.

### (Configuration 8)

The information processing device any one of configurations 1 to 7, wherein the storage unit stores the information about the cell, the information about the detachment condition, and information about a culture condition for the cell in association with one another.

### (Configuration 9)

The information processing device according to configuration 8, wherein information about the culture condition includes at least one piece of information selected from the group consisting of cell seeding density information, information about a culture medium, information about a culture duration, and information about a culture environment.

### (Configuration 10)

A cell detachment system comprising:
the information processing device according to any one of configurations 1 to 9; and
a detachment device configured to detach the cell adhering to the culture substrate from the culture substrate by applying an external force to the culture substrate.

### (Configuration 11)

A cell detachment system comprising:
a measurement device configured to perform a measurement to acquire information about the cell; and
the information processing device according to any one of configurations 1 to 9.

### (Configuration 12)

The information processing device according to any one of configurations 1 to 9, further comprising a display control unit configured to perform control to cause a display unit to display a driving state of a detachment device configured to detach the cell adhering to the culture substrate from the culture substrate by applying an external force to the culture substrate.

### (Configuration 13)

The information processing device according to configuration 12, wherein the driving state includes information about a temporal change in the driving state of the detachment device.

### (Configuration 14)

The information processing device according to configuration 12 or 13, wherein the display control unit performs control to cause the display unit to display a driving condition for the detachment device.

(Configuration 15) information processing device according to configuration 12, wherein the display control unit performs control to cause the display unit to display the driving state in association with a driving condition for the detachment device.

### (Method 1)

An information processing method, comprising:
acquiring information about a cell;
acquiring information about a detachment condition for detaching the cell adhering to a culture substrate from the culture substrate by an external force to be applied to the culture substrate; and
storing the information about the cell and the information about the detachment condition in association with each other.

The information processing device 1000 and the information processing method according to the exemplary embodiments of the present disclosure store information about cells and information about detachment conditions in association with each other, thereby making it possible to easily recognize the detachment conditions under which the cells have been detached.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-115612, filed Jul. 19, 2024, which is hereby incorporated by reference herein in its entirety.

What is claimed is:

1. An information processing device, comprising:
at least one memory storing instructions; and
at least one processor that, upon execution of the stored instructions, is configured to operate as:
a cell information acquisition unit configured to acquire information about a cells;
a detachment condition acquisition unit configured to acquire information about a detachment condition for detaching the cell adhering to a culture substrate from the culture substrate by an external force being applied to the culture substrate; and
a storage unit configured to store the information about the cell and the information about the detachment condition in association with each other.

2. The information processing device according to claim 1, wherein vibration is applied to the culture substrate to apply the external force to the culture substrate.

3. The information processing device according to claim 2, wherein the information about the detachment condition includes one or more or all pieces of information selected from the group including of information about an intensity of the vibration to be applied to the culture substrate, information about a frequency of the vibration to be applied to the culture substrate, and/or information about a duration of vibration to be applied to the culture substrate.

4. The information processing device according to claim 2,
wherein the vibration to be applied to the culture substrate is generated by application of an ultrasonic wave generated by an ultrasonic transducer, and
wherein information about an intensity of the vibration to be applied to the culture substrate includes information about a voltage to be applied to the ultrasonic transducer.

5. The information processing device according to claim 1, wherein the information about the detachment condition includes one or more or all pieces of information selected from the group including of information about detachment solution to be used to detach the cell from the culture substrate, and information about an acoustic matching material.

6. The information processing device according to claim 1,
wherein the information about the detachment condition is information obtained based on a detachment result of detaching the cell from the culture substrate, and
wherein the information about the detachment result includes one or more or all pieces of information selected from the group including of information about a detachment rate of the cell, information about a survival rate of the detached cell, information about a growth rate of the detached cell, and information about functionality of the detached cell.

7. The information processing device according to claim 1, wherein the information about the cell includes information about cell type.

8. The information processing device according to claim 1, wherein the storage unit stores the information about the cell, the information about the detachment condition, and information about a culture condition for the cell in association with one another.

9. The information processing device according to claim 8, wherein information about the culture condition includes one or more or all pieces of information selected from the group consisting of cell seeding density information, information about a culture medium, information about a culture duration, and information about a culture environment.

10. A cell detachment system comprising:
at least one memory storing instructions; and
at least one processor that, upon execution of the stored instructions, is configured to operate as:
a cell information acquisition unit configured to acquire information about a cells;
a detachment condition acquisition unit configured to acquire information about a detachment condition for detaching the cell adhering to a culture substrate from the culture substrate by an external force being applied to the culture substrate; and
a storage unit configured to store the information about the cell and the information about the detachment condition in association with each other; and
a detachment device configured to detach the cell adhering to the culture substrate from the culture substrate by applying an external force to the culture substrate.

11. A cell detachment system comprising:
a measurement device configured to perform a measurement to acquire information about the cell;
a cell information acquisition unit configured to acquire information about a cells;
a detachment condition acquisition unit configured to acquire information about a detachment condition for detaching the cell adhering to a culture substrate from the culture substrate by an external force being applied to the culture substrate; and
a storage unit configured to store the information about the cell and the information about the detachment condition in association with each other.

12. The information processing device according to claim 1, wherein execution of the stored instructions further configures the at least one processor to operate as further comprising a display control unit configured to perform control to cause a display device to display a driving state of a detachment device configured to detach the cell adhering to the culture substrate from the culture substrate by applying an external force to the culture substrate.

13. The information processing device according to claim 12, wherein the display of the driving state includes displaying information about a temporal change in the driving state of the detachment device.

14. The information processing device according to claim 12, wherein the display control unit performs control to cause the display unit to display a driving condition for the detachment device.

15. The information processing device according to claim 12, wherein the display control unit performs control to cause the display unit to display the driving state in association with a driving condition for the detachment device.

16. An information processing method for an information processing apparatus that includes processor and a memory containing instructions that, when executed by the processor, cause the processor to execute the control method comprising:
acquiring information about a cell;
acquiring information about a detachment condition for detaching the cell adhering to a culture substrate from the culture substrate by an external force to be applied to the culture substrate; and
storing the information about the cell and the information about the detachment condition in association with each other.
